(19) United States
(12) Patent Application Publication
(10) Pub. No.: US 2007/0049910 A1
(43) Pub. Date: Mar. 1, 2007

(54) Eye-safe photocosmetic device

(75) Inventors: Gregory B. Altshuler, Lincoln, MA (US); Henry H. Zenzie, Dover, MA (US); Christopher Gaal, Mansfield, MA (US); Robert R. Lopez, Boxford, MA (US); Guangming Wang, Bedford, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 11/500,588

(22) Filed: Aug. 8, 2006

Related U.S. Application Data
(60) Provisional application No. 60/706,505, filed on Aug. 8, 2005.

(51) Int. Cl. A61B 18/18

(52) U.S. Cl. 606/9

(57) ABSTRACT

Devices and methods for treating tissue with radiation, including light and other optical radiation, in a manner that is eye-safe are described. In one embodiment, a photocosmetic treatment device has a cavity into which tissue to be treated is drawn. The device determines whether the tissue is safe to treat and whether the tissue may be tissue associated with the eyes, such as an eyelid. In another embodiment, an eye-safe pulse of radiation is provided at a time interval prior to treatment of the tissue. The pulse is at a wavelength of radiation that the human eye perceives as particularly intense and uncomfortable, even though the pulse is not dangerous or destructive. If the device is oriented to treat eye tissue, directly or through the eyelid, the pulse will cause an aversive reaction in the subject being treated that inhibits the treatment.

### REFERENCE TO RELATED APPLICATION

This application claims priority to U.S. Provisional Application No. 60/706,505, filed Aug. 8, 2005.

### BACKGROUND OF THE INVENTION

1. Technical Field.

The invention relates to the photocosmetic treatment of skin. In particular, the invention relates to eye safe, efficacious, devices for treating skin.

2. Background Art

There exists a variety of conditions treatable using photocosmetic procedures (also referred to herein as photocosmetic treatments), including light-based (e.g., using a laser, lamp or other light source) hair growth management, treatment of pseudofolliculitis barbae, treatment of acne, treatment of various skin lesions (including pigmented and vascular lesions), leg vein removal, tattoo removal, facial resurfacing, treatment of fat, including cellulite, removal of warts and scars, and skin rejuvenation, including treatment of wrinkles and improving skin tone and texture, and various other dermatology treatments.

Currently, various photocosmetic procedures are performed using professional-grade devices that cause destructive heating of target structures located in the epidermis/dermis of a patient's skin. These procedures are typically performed in a physician's office or the office of another licensed practitioner, partially because of the expense of the devices used to perform the procedures, partially because of safety concerns related to the devices, and partially because of the need to care for optically induced wounds on the patient's skin. Such wounds may arise from damage to a patient's epidermis caused by the high-power radiation and may result in significant pain and/or risk of infection.

While certain photocosmetic procedures, such as CO₂ laser facial resurfacing, will continue to be performed in the dermatologist's office for medical reasons (e.g., the need for post-operative wound care), there are a large number of photocosmetic procedures that could be performed in either a medical or in a non-medical environment (e.g., home, barber shop, or spa), if the consumer could perform the procedure in a safe and effective manner. Even for procedures performed in a medical environment, less expensive, safer and easier to use devices would be advantageous and reduced skin damage would reduce recovery time.

Photocosmetic devices for use in medical or non-medical environments preferably should be designed to be safe for use on the skin or other tissues, and, for example, to prevent eye and skin injuries, including damage to a patient's iris even when an eye lid is closed. Such devices also preferably should be designed to be easy to use, thus allowing an operator to achieve acceptable cosmetic results with only simple instructions and potentially to enhance the overall safety of the device. The safety of currently available photocosmetic devices, including those used in the professional setting, could be improved in these areas.

For example, eye-safe consumer devices would prevent accidental injuries to users of those devices. Prior art solutions to provide eye safety generally have been directed to protecting the retina and may not protect a patient's iris. The iris often includes a high concentration of melanin which may absorb treatment energy even when the eye lid is closed. Often eye protection techniques (e.g. frosted glass, defocused optics, low power) negatively impact the efficacy of treatment. Furthermore, existing devices sold to consumers are generally of very low power, and the safety measures on such devices may not adequately protect the retina, iris or any other part of the eye or other tissue when used in conjunction with a consumer device designed to irradiate tissue using higher power densities and fluences.

It would be desirable to provide a skin treatment device which provides increased eye safety and efficacy.

### SUMMARY OF THE INVENTION

One aspect of the invention is a method for treating tissue of a subject with radiation in an eye-safe manner. The tissue may be irradiated with eye-safe radiation having a wavelength and intensity that cause an aversive response by the subject when the eye-safe radiation irradiates the subject's eye. After the eye-safe radiation is transmitted, there is a pause for a predetermined period of time to see if any aversive reaction occurs. If no aversive reaction occurs, the tissue is irradiated with an appropriate treatment radiation. If an aversive reaction does occur, the tissue is not irradiated with the treatment radiation.

Preferred embodiments of this aspect may include some of the following additional features. The eye-safe radiation may have a wavelength in the range of 600-680 mn, and may have a wavelength that is predominately red. The eye-safe radiation may have an intensity in the range of 1-10 mW/cm². The period of time may be in a range of approximately 0.1 to 3.0 seconds, or more particularly may be in a range of approximately 1.0 to 2.0 seconds.

The method may further include determining whether the aversive response has occurred and inhibiting the transmission of the treatment radiation when the aversive response has occurred. Alternatively, the method may rely on the aversive response to ensure that no treatment radiation is applied to the eye. The method may further include contacting the tissue with an applicator to transmit the eye-safe radiation, and irradiating with the eye-safe radiation only if the applicator is in contact with the tissue. The method may further include irradiating the tissue with the treatment radiation only if the applicator is in contact with the tissue. The method may also include orienting an applicator to irradiate the tissue with the eye-safe radiation, and irradiating the tissue only if the applicator is in proximity of the tissue.

Another aspect of the invention is an apparatus for treating tissue with radiation in an eye-safe manner, which includes a controller for controlling the production of radiation and configured to provide first and second control signals; a first radiation source configured to produce in response to the first control signal an eye-safe radiation at an intensity that irritates a subject's eye; a second radiation source configured to produce in response to the second control signal a treatment radiation; a radiation transmission path configured to transmit radiation from the first radiation source to the tissue through a radiation transmission surface; a sensor in electrical communication with the controller and configured to provide a sensor signal when the radiation transmission surface is in proximity to the tissue. The controller may be configured to provide the second control signal after a predetermined time interval following the first control signal and when the sensor signal indicates that the radiation transmission surface remains in proximity to the tissue.

Preferred embodiments of this aspect of the invention may include some of the following additional features. The first radiation source may be a diode. The first radiation source may be configured to produce radiation in the range of 600-680 nm. The first radiation source may be configured to produce radiation having a wavelength that is predominately red. The first radiation source may be configured to produce radiation having an intensity in the range of 1-10 mW/cm².

The predetermined time interval may be in the range of approximately 0.1 to 3.0 seconds, or, more particularly, may be in the range of approximately 1.0 to 2.0 seconds. The controller may be configured to provide the second control signal when the radiation transmission surface is in contact with the tissue, and may be configured to provide the first control signal when the radiation transmission surface is in contact with the tissue. The sensor may be configured to detect an aversive response from the subject in response to the eye-safe radiation. The aversive response may be any movement that causes the subject to move the device from the tissue, or any movement that indicates to a person treating the subject that the eye may be irradiated, including, without limitation, squinting, pupil dilation, eye movement, head movement, and arm movement.

The first radiation source may be further configured to provide sensor radiation. The sensor may be a detector configured to detect the sensor radiation. The sensor radiation may have a wavelength in the near infrared range. The detector may be configured to provide the sensor signal when the sensor radiation exceeds a first predetermined threshold.

The radiation transmission path may be configured to substantially totally internally reflect the sensor radiation when the radiation transmission surface is not in contact with the tissue. The radiation transmission path may be configured to substantially totally internally reflect the eye-safe radiation when the radiation transmission surface is not in contact with the tissue.

The radiation transmission path may be configured to substantially totally internally reflect the treatment radiation when the radiation transmission surface is not in contact with the tissue. The radiation transmission path may further include a first waveguide section; a second waveguide section; and a diffuser. The first waveguide section may be located between the first source and the diffuser and the second waveguide section may be located between the diffuser and the radiation transmission surface. The diffuser may extend across substantially the entire the radiation transmission path, and may be made of plastic, glass, sapphire or other suitable material. The second waveguide section may be sapphire or other suitable material, and may include a cooling mechanism configured to cool the tissue.

Another aspect of the invention is an apparatus for treating tissue with radiation in an eye-safe manner. The apparatus may include a radiation source assembly in electrical communication with a controller; a waveguide configured to transmit radiation from the radiation source assembly to the tissue; and a sensor in electrical communication with the controller and configured to provide a sensor signal when the radiation transmission surface may be in proximity to the tissue. The radiation source assembly may be configured to provide in response to signals from the controller a first radiation that may be eye-safe and of an intensity capable of causing an aversive reaction from a subject when irradiating the subject's eye. The radiation source assembly may also be configured to provide, a predetermined time after the first radiation, a second radiation capable of treating the tissue. The second radiation may only be provided when the sensor indicates that the waveguide remains in proximity of the tissue.

Preferred embodiments of this aspect of the invention may include some of the following additional features. The radiation source assembly may be further configured to provide a third radiation, and the sensor may be configured to detect the third radiation and issue a sensor signal based on the level of radiation detected.

The waveguide may include a diffuser extending across a portion of the waveguide and oriented to diffuse radiation produced by the radiation source assembly. The waveguide may include a cooling mechanism configured to cool the tissue. The sensor may be configured to provide a sensor signal only when the radiation transmission surface is in contact with the tissue.

Another aspect of the invention is an apparatus for photocosmetic treatment of a subject's tissue, which may include a pressure source; a cavity having an open end, the cavity in fluid communication with the pressure source, and the open end configured to receive the tissue when the pressure source applies pressure; at least one radiation source configured to transmit radiation into the cavity; and a sensor configured to issue a sensor signal. The sensor signal may prevent the transmission of radiation from the radiation transmission source when the sensor detects tissue that may be not suitable for treatment.

Preferred embodiments of this aspect of the invention may include some of the following additional features. The radiation source may be configured to transmit radiation from at least two different directions within the cavity. The radiation source may be configured to treat a set of two or more volumes of tissue each separated by untreated tissue. The radiation source may be configured to provide radiation to an array of independent treatment sites within the cavity, wherein each such treatment site may be separated by untreated tissue within the cavity.

The sensor may be a pressure sensor. The sensor may be a depth sensor configured to sense a depth of the tissue within the cavity, and may be configured to provide a control signal inhibiting the transmission of radiation by the radiation source when the tissue extends beyond a predetermined depth into the cavity. The sensor may be a radiation intensity sensor, and may be configured to provide a control signal inhibiting the transmission of radiation by the radiation source when the radiation exceeds a predetermined threshold. The sensor may be configured to provide a control signal inhibiting the transmission of radiation by the radiation source when the radiation is substantially totally internally reflected.

The apparatus may be configured to operate within a predetermined safety ratio. The cavity may have a depth that is greater than the depth of a target in the tissue to be treated, when measured from the target to the surface of the tissue, and may have a side that is less than four times the depth of a target in the tissue when measured from the target to the surface of the tissue.

The radiation source may be configured to irradiate the tissue at a fluence of about 0.1 to about 100 J/cm². The radiation source may be configured to irradiate the tissue at a pulse width of about 1 ms to about 500 ms. The radiation source may be configured to irradiate the tissue at a wavelength range between approximately 400-1350 nm, and, more particularly, at a wavelength range between approximately 600-1200 nm.

Another aspect of the invention is a method for photocosmetic treatment of a subject's tissue that may include drawing a volume of the tissue into a cavity; determining whether the volume of tissue may be safe to treat using radiation; and treating the volume of tissue with radiation based on the determination. The volume of tissue is not treated, if it is determined that the tissue may be unsafe to treat, and is treated if it is determined that the tissue is safe to treat.

Preferred embodiments of this aspect of the invention may include some of the following additional features. Treating may include transmitting radiation from at least two different directions, and the radiation from at least two different directions may overlap at one or more targets on the skin. The radiation from at least two different directions may treat a set of two or more volumes of tissue each surrounded by untreated tissue.

The method may also include providing an array of independent treatment sites within the volume of tissue, in which each such treatment site is separated by untreated tissue within the volume. A pressure applied to the tissue may be sensed to determine whether it is safe to treat the tissue. The depth of the volume of the tissue within the cavity may be sensed so that it can be determined whether the tissue is safe to treat. The determination may also be made by sensing radiation using a radiation intensity sensor.

The treatment may be at a fluence of about 0.1 to about 100 J/cm², a pulse width of about 1 ms to about 500 ms, and within a wavelength range of approximately 400-1350 nm, or, more particularly, approximately 600-1200 nm.

### BRIEF DESCRIPTION OF THE DRAWINGS

Illustrative, non-limiting embodiments of the present invention will be described by way of example with reference to the accompanying drawings, in which the same reference numeral is for the common elements in the various figures, and in which:

FIG. 1 is a cross-sectional perspective view of a photocosmetic device according to some aspects of the present invention;

FIG. 2 is a cross-sectional perspective side view of the treatment head of the device of FIG. 1;

FIG. 3 is a cut away side view of the device of FIG. 1;

FIGS. 4A and 4B are schematic views showing the optical properties and additional safety features of the device of FIG. 1;

FIG. 5A is a light distribution chart for a prior art direct light treatment device;

FIG. 5B is a light distribution chart of one example of closed loop light coupling according to the invention;

FIG. 6 is a graph of skin fold height vs. pressure for a cavity similar to the cavity of FIG. 1;

FIG. 7A is a light distribution chart for closed loop light coupling without a reflector for a treatment head similar to the treatment head of FIG. 1;

FIG. 7B is a light distribution chart for closed loop light coupling with a reflector for a treatment head similar to the treatment head of FIG. 3;

FIG. 8A is a light intensity distribution chart along a width axis for a cavity similar to the cavity of FIG. 1 and having a width of 5 mm;

FIG. 8B is a light intensity distribution chart along a height axis for a cavity similar to the cavity of FIG. 1 and having a width of 5 mm;

FIG. 9A is a light intensity distribution chart along a width axis for a cavity similar to the cavity of FIG. 1 and having a width of 4 mm;

FIG. 9B is a light intensity distribution chart along a height axis for a cavity similar to the cavity of FIG. 1 and having a width of 4 mm;

FIG. 10A is a side view of another embodiment of the invention including a flashlamp optical radiation source;

FIG. 10B is a side view of yet another embodiment of the invention including a flashlamp integrated with radiation directing elements;

FIG. 11 is a side view of a pressure controlled firing mechanism to protect a person's eye according to another aspect of the invention;

FIG. 12 is a perspective view of a hair growth management device including a self contained power supply according to another embodiment of the invention;

FIG. 13 is a perspective view of a conical shaped prism for use in yet another embodiment of the invention; and

FIG. 14 is a schematic view of an alternate embodiment of an eye-safe treatment device.

### DETAILED DESCRIPTION

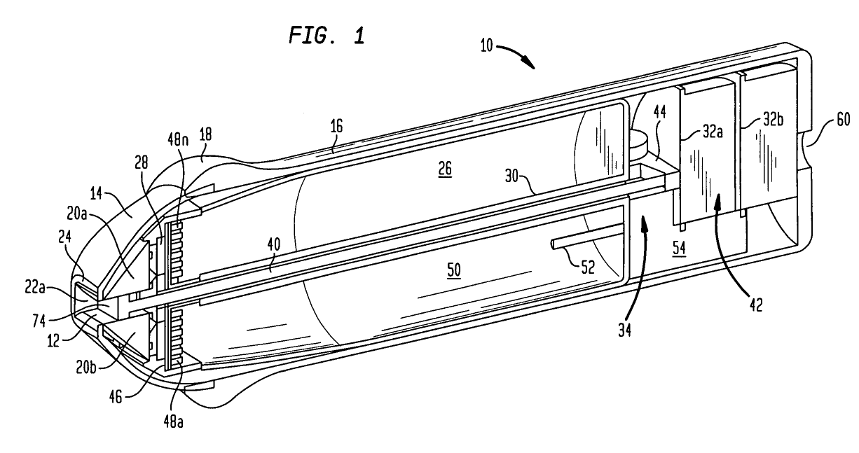

The embodiments described below provide improved optical radiation delivery and safety. For example, with reference to FIG. 1, a device 10 includes a cavity into which skin is drawn, and light delivery mechanisms to direct light to the skin within the cavity from multiple directions. The skin preferably is placed in the cavity by applying negative pressure, but other methods are possible, such as positive pressure or crimping the tissue within the cavity or a channel. By optimizing the dimensions of the cavity, the optical radiation from two or more different directions may be overlapped or combined at the location of one or more targets within the skin to be treated. This combined treatment energy within the skin increases the efficacy of treatment while also improving the safety ratio to better protect the epidermis. The safety ratio is the ratio of the temperature change of the treatment target over the temperature change of the epidermis. Generally, it is preferable to have a high temperature at the target without damaging the epidermis (i.e., excessive temperature at the epidermis). Combining light from multiple directions means that targets within the tissue receive light from more than one direction while the skin surface receives light substantially from only one direction. As a result, the target receives more light than the skin surface.

In addition, drawing the skin into the cavity compresses the skin thereby removing blood and thinning the skin within the cavity. Since blood often absorbs optical radiation at the wavelengths used for treatment of the target, removing blood improves efficacy by avoiding energy loss to blood absorption which increases the energy available for absorption by the real target. Further, removal of blood from the skin within the cavity also increases safety by avoiding bulk heating due to blood absorption of optical radiation. Thinning the skin decreases scattering of the optical radiation and the distance to the target both of which improve efficacy.

Drawing the skin into the cavity also stretches the skin, which stretches the basal membrane. Stretching the basal membrane can decrease the melanin optical density (MOD). Like blood, the melanin in the basal membrane often absorbs optical radiation at the wavelengths used for treatment. Consequently, like removing blood, decreasing the MOD provides more energy for absorption by the target thereby increasing efficacy and also reduces bulk heating that can lead to skin damage thereby increasing safety.

Directing light to skin within the cavity can also provide eye safety. In one embodiment, light traveling substantially parallel to the opening of the cavity is delivered to the skin within the cavity such that there is little or no direct light emission from the cavity. Direct light presents a potential risk of eye injury because such direct light can be focused onto the retina or absorbed by melanin in the iris thereby (if the intensity is sufficient) damaging the eye. Accordingly, reducing or eliminating direct light emission from the cavity improves eye safety. The iris can also be damaged by absorption of light propagating through a closed eye lid. Thus, reducing or eliminating direct light emission from the cavity also reduces the amount of light that can propagate through the eye lid and be absorbed by the iris.

To further improve eye safety, the device may only direct light within the cavity when it is determined that skin is within the cavity. Many mechanisms for making such a determination are possible. In one embodiment, the light delivery mechanisms may have total internal reflection such that light will not pass into the cavity until skin comes into contact with the internal walls of the cavity. In another embodiment, one or more sensors are located near the opening of the cavity and will not allow light to be delivered to the cavity until they detect the presence of skin drawn within the cavity. As another eye safety measure, the cavity may be blocked, for example, by a shutter, when negative pressure is not being applied to the cavity and such shutter may only open when skin is drawn within the cavity.

Another safety mechanism involves detecting skin which is not sufficiently firm, for example, skin around the eye area, including the eye lid, and not permitting the device to emit light into the cavity when such skin is detected. Because the skin around the eye, especially the eye lid, is very thin, treating around the eye area can lead to light propagation through the skin, absorption by the iris and potential eye damage. To prevent such injury, one or more sensors can be located within the cavity at a height—that is, distance from the cavity opening—beyond which other skin of typical firmness can be drawn for the given dimensions of the cavity. As a result, if these sensors detect the presence of skin, it is an indication that the skin is of a type that should not be treated (e.g., skin around the eye), and the sensors prevent the device from emitting light into the cavity.

Eye safety can also be improved by requiring that a certain level of pressure be applied by the device to the skin before the device will emit light into the cavity. That is, the device will not emit light into the cavity until it is pushed sufficiently hard against the skin. One mechanism for this is a pressure controlled firing mechanism. This can provide eye safety because such pressure may not be applied to the eye, or at least not without significant pain, such that the device cannot emit light into the cavity if it is directed toward the eye.

Eye safety can also be improved by utilizing light to create an aversive reflex in the subject being treated. In yet another embodiment, light having a wavelength that is generally eye-safe but that is particularly irritating to the eye is transmitted at a level that does not cause damage, but that is intense enough to cause an aversive reaction or reflex, such as the closing of the eye, turning of the head, or movement of the device away from the face.

Treatment may provide a result that is either permanent or temporary (e.g., permanent hair reduction or temporary hair removal) and the result may be immediate (e.g., vaporization of all or a portion of a hair shaft or change in the structure of a hair shaft) or take time to manifest (e.g., a hair shaft eventually falls out). In addition, multiple treatments may be required to provide a result, for example, a result may require the accumulated effects of radiation treatment, and again such result might be permanent or temporary. If temporary, periodic treatments may be required to maintain the result. For example, several treatments might be required to remove hair, and to maintain hair-free skin, periodic re-treatment may be required. Although hair removal was given as an example of treatment, it is to be understood that many dermatologic and other treatments are possible. The devices disclosed herein may be used to treat various targets within skin, including but not limited to hair follicles, sebaceous glands, wrinkles, scars, deep dermis, dermis/hypodermis junction, subcutaneous fat and superficial muscle, by modifying the dimensions of the cavity and other parameters. Certain embodiments may also be useful for other treatments or devices in which eye safety may be a concern. For example, in a consumer device to treat dental tissue, it may be appropriate or beneficial to ensure that the light cannot be shined accidentally or otherwise in a subject's eyes, even though the device is not intended for use near or around the eyes.

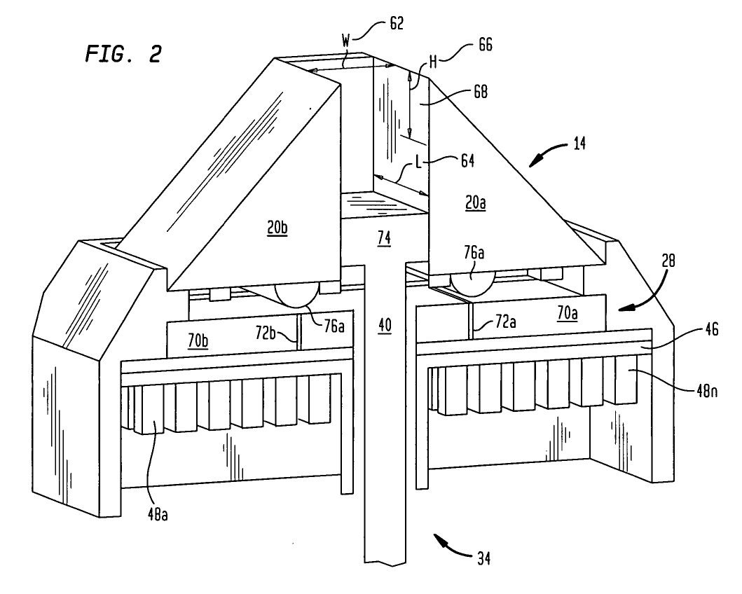

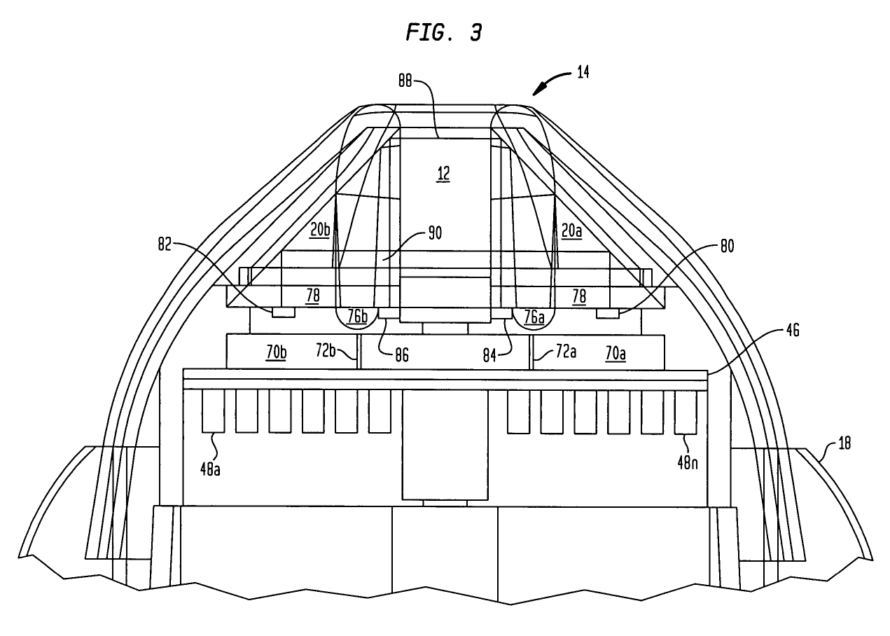

Now referring to FIG. 1, an exemplary device 10 can be used to treat tissues in a person's skin including, for example, hair follicles for hair growth management, including hair removal. The device 10 includes a housing 16 having a curved section 18 for easier handling. Device 10 further includes a treatment head 14 disposed adjacent the housing 16 includes two radiation directing elements 20a and 20b (generally referred to as radiation directing elements 20) and sidewalls 22a and 22b (not shown) which form a cavity 12. The treatment head 14 further includes an optical radiation source 28 optically coupled to the radiation directing elements 20 as described below in more detail in conjunction with FIGS. 2 and 3.

The treatment head 14 includes an outer edge 24, which may be contoured to form a more efficient pressure seal with the skin. In order to provide cooling to the radiation source 28, device 10 includes a chamber 26 that includes a volume of liquid or other material 50. Liquid or other material 50 is thermally coupled to the radiation source 28 and optionally the radiation directing elements 20. The device 10 optionally includes a dispenser (not shown) which can dispense, for example, a cooling lotion, to the skin. Device 10 also includes a skin gathering implement 34 having a cylinder 30 enclosing a piston rod 40 coupled to a piston 74 to provide a manually generated source of differential pressure (e.g. low or negative pressure or vacuum) within the cavity 12 to draw skin within the cavity. The piston forms a moveable pneumatic seal with the radiation directing elements 20 and sidewalls 22a and 22b. The skin gathering implement 34 further includes a reversing mechanism 44 coupled to rod 40.

In alternative embodiments, each radiation directing element 20a and 20b can include multiple radiation directing elements and sidewalls 22a and 22b can also include one or more radiation directing elements. In an alternative embodiment, the optical radiation source 28 may be located remotely in a console and optically coupled to the treatment head 14, for example, through a fiber optic cable. In another alternative embodiment, the optical radiation source 28 may be located elsewhere within device 10.

In all embodiments, optical radiation source 28 may include one or more optical radiation sources, which may be any of a number of different types, including, without limitation, both broad and narrow band light sources such as lasers, diode lasers, tunable lasers, diodes, halogen lamps, flashlamps, and/or other types of lamps. Further, several different sources and/or types of sources can be used to provide radiation at various wavelengths. In one embodiment the optical radiation source 28 can include multiple sources, for example, light emitting diodes (LEDs), lamps, laser diode bars, lasers, and other sources. One optical radiation source can provide radiation to one or more radiation directing elements, or multiple radiation sources can provide radiation to one or more radiation directing elements. For example, a beam splitter or other means known in the art may be used to direct light from one source to multiple radiation directing elements. In one embodiment the radiation directing elements 20 are provided as prisms, for example triangular equilateral prisms or right angle prisms. However, various other shapes are possible.

A cooling system of device 10 includes a heat sink 46 having fins 48a-48n that are in thermal contact with cooling material 50 disposed within chamber 26. Cooling material 50 may be a phase transfer material which changes phases as it absorbs heat from heat sink 46 or cooling material 50 may be a liquid which is circulated within chamber 26 through conductor pipe 52 coupled to pump 54. Optionally, device 10 may include batteries (not shown) or a connection port 60 disposed in the housing 16 can provide a connection (not shown) to an external power source, such as a wall outlet. Optionally, device 10 may include a mechanism for chilling material 50 when it is circulated within chamber 26 or connection port 60 may be used to connect chamber 26 through pipe 52 with an external source (not shown) of cooling material 50, for example, water. Device 10 includes a controller 42, e.g., electronic circuit boards 32a and 32b disposed within the housing 16, which may include control circuitry coupled to optical source 28, sensors, and other components as described below.

Gathering or drawing skin into the cavity 12 changes the optical properties of skin to be treated. Drawing the skin within cavity 12 compresses the skin and causes both a stretching of the skin and also a restriction of the blood flow within the skin in the cavity. Advantageously the irradiance of a target within the skin in the cavity, for example, a follicle including a hair bulb or a sebaceous gland, can be increased, for example, by a factor of 1.2-2.5, because of decreased scattering, amount of blood, and skin thickness of the skin within the cavity.

In operation, device 10 is operated with a stamping motion or sliding motion to treat skin. A stamping motion is accomplished, for example, by placing the device 10 in contact with the skin and treating the skin, then removing the device from the skin and placing the device in contact with another area of the skin. A sliding motion is accomplished by simultaneously moving the device over the skin surface as the device treats the skin. The treatment of the skin can be coordinated with the velocity of the movement of the device over the skin surface.

As the treatment head 14 is placed in contact with the surface of the skin, a portion of the skin to be treated is drawn into the cavity 12 by activating skin gathering implement 34 to lower pressure within the cavity 12. The skin within the cavity is then exposed to treatment radiation from optical source 28 through radiation directing elements 20a and 20b.

In one embodiment, as device 10 is placed in contact with skin, pressure of skin against piston 74 is sensed by the reversing mechanism 44, which then pulls the rod 40 within the cylinder 30 away from the entrance to the cavity 12 such that piston 74 is also moved away from the entrance. The movement of piston 74 generates the pressure differential within the cavity 12, which pulls the skin within the cavity. Piston 74, thus, acts as a shutter for cavity 12 when device 10 is not in contact with skin.

As an alternative to skin gathering implement 34, device 10 could include an external vacuum source (not shown) coupled to the cavity 12. This vacuum source, as well as skin gathering implement 34 may be triggered by a button (not shown) pressed by an operator on device 10 or, as explained above, pressure of skin against piston 74 or another type of shutter might be used to trigger the activation of the external vacuum source or skin gathering implement.

The dimensions of cavity 12 are selected such that targets in the skin drawn into cavity 12 receive optimal treatment from light passing into cavity 12 from different directions through radiation directing elements 20a and 20b. For example, hair follicles or sebaceous glands within the skin may be substantially centered between radiation directing elements 20a and 20b, and the optical radiation applied to the skin can be selected to overlap in the central portion of the cavity such that the targets receive light from both radiation directing elements while the skin surfaces against the radiation directing elements receive light only from the radiation directing element with which they are in contact.

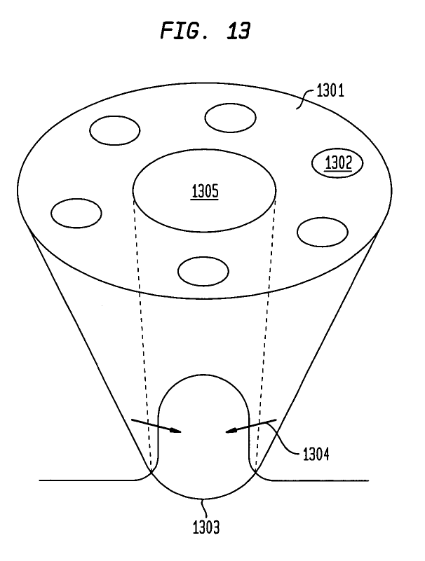

In device 10, the radiation directing elements are located on opposite sides of cavity 12. However, in alternative embodiments the radiation elements may be located on adjacent sides such that they are at a ninety degree angle from each other. As mentioned above, one or more radiation directing elements may be located on each side of cavity 12. In addition, cavity 12 may be circular instead of square or rectangle or any other shape. For example, referring to FIG. 13, instead of radiation directing elements 20a and 20b, device 10 can include a conical shaped prism 1301 having a cylindrical hole 1305 that serves as the cavity into which skin 1303 is drawn with negative pressure. In addition, a plurality of light sources 1302 (e.g., laser diodes, LEDs, lamps) may be coupled to conical prism 1301 to direct light beams 1304 into the portion of the skin 1303. Conical prism 1301 can provide axial symmetry allowing for higher amplification of light inside skin than the planar symmetry provided by radiation directing elements 20.

The dimensions of cavity 12 as well as the amount of negative pressure (also referred to as pressure differential) that may be applied are selected in accordance with the amount of skin to be treated and desired effect on both mechanical and optical properties of skin while being treated. For most applications, it is desirable to treat a large amount of tissue with each application of light such that less time is required to complete the overall treatment of a larger area of skin. However, this is contrasted with also desiring smaller, less expensive devices and other factors. For example, the amount of skin that can be treated at one time by device 10 is limited by the amount of skin that can be drawn into cavity 12. Although the width (shown as W 62 in FIG. 2) of cavity 12 can be made quite large to treat more skin, doing so can prevent radiation from radiation directing elements 20a and 20b from overlapping and combining within the skin in cavity 12. If device 10 further includes radiation directing elements in side walls 22a and 22b, then the same is true for the length(shown as L 64 in FIG. 2) of cavity 12. Similarly, making these dimensions too small can cause overlap of the radiation and the skin surface. Thus, the length and width of cavity 12 are limited by the desire to combine radiation from different radiation directing elements delivering radiation from different directions into cavity 12.

In addition, the height (shown as H 66 in FIG. 2) of cavity 12 is also limited. In theory, a very long or deep height could be used to draw more skin into cavity 12 for treatment. However, for any given dimensions at the entrance to the cavity, only a certain amount of skin may be drawn into the cavity without bruising the skin. Thus, the height dimension is also limited.

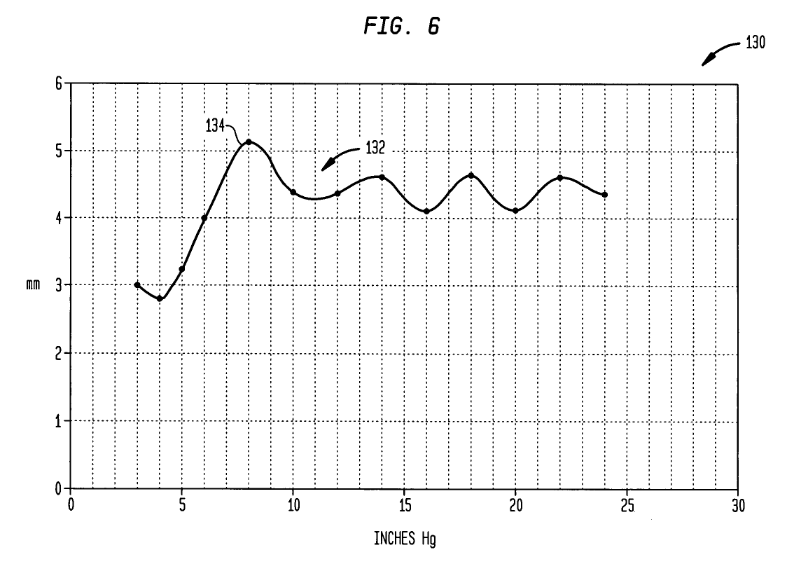

In the embodiment shown in FIG. 1, the cavity 12 of device 10 has a length L 64 of approximately 10 mm which matches the optical sources 28 having lengths of approximately 10 mm. In this embodiment, the cavity 12 has a width W 66 of about 2 mm to about 6 mm, and preferably about 4 mm. This width and length allows firm treatable skin (e.g., not skin around the eye area) to be treated with combined uniform radiation from multiple radiation directing elements 20 in the predetermined volume V 68. In this embodiment, the height of the cavity is 13 mm and a pressure differential of approximately 20 cm of Hg (8 inches of Hg) is applied. This is described further in conjunction with FIG. 6.

In one embodiment, the targets of the treatment are hair follicles. Typically hair follicles are found at a skin depth of 1-4 mm. As a result, gathering skin within cavity 12 such that the height (Hₛₖᵢₙ 66 shown in FIG. 2) of the skin within the chamber is about 2 mm to about 6 mm. Such a skin height hₛₖᵢₙ 66 locates the person's hair follicles within the predetermined volume V 68 (FIG. 2) and subjects the follicles to combined radiation from the radiation directing elements 20 coupled to the optical radiation source 28.

In another embodiment for treating acne, the targets of the treatment are sebaceous glands. Typically sebaceous glands are found at a skin depth of 1-3 mm. As a result, gathering skin within cavity 12 such that the height of the skin within the chamber is about 1 mm to about 3 mm locates sebaceous glands within the predetermined volume and subjects the sebaceous glands to combined radiation from the radiation directing elements 20 coupled to the optical radiation source 28.

Optionally a lotion may be applied to the skin to allow the skin to be more easily drawn within the cavity. Such a lotion can also improve optical and thermal coupling between the skin and the internal walls of the cavity.

As described above, as the skin is drawn into cavity 12, blood is removed. This allows the use of wavelengths that are normally absorbed by blood to be used more effectively. For example, optical sources 28 may generate optical radiation from 380-1350 nm. While drawing skin within cavity 12 may remove most of the blood within the skin, it may concentrate the remaining blood in the skin at the top of the cavity—that is, in the tip of the fold of the skin that is deepest or at the greatest height within cavity 12. Such concentration of blood may be the focus of treatment for removal of superficial targets such as vascular lesions.

Furthermore, the temperature of epidermis can be decreased by a factor of about 1.1 to about 1.5 times because the basal membrane is stretched thereby decreasing the melanin optical density (MOD) which, as described above, can absorb part of the treatment energy. Consequently, the tissue (e.g. hair follicle) can be treated more effectively as a result of the optical and mechanical property changes created in the skin as it is drawn into cavity 12 and from the combined optical radiation from multiple radiation directing elements 20 which

Now referring to FIG. 2, further details of the device 10 are shown. The cavity 12 includes a length L 64 and a width W 62. It will be appreciated, that in other embodiments, device 10 could include a cavity having a different geometry, for example, circular, square, hexagonal, asymmetric, triangular, domed, and instead of straight internal walls, such walls could be, for example, slanted (inwardly or outwardly), curved, or made of flexible or soft material. A skin height hₛₖᵢₙ 66 within the chamber is measured from the entrance of the cavity 12 as shown. The cavity 12 includes a volume 68 in which radiation from radiation directing elements 20a and 20b is combined. In this embodiment, the skin gathering implement 34 includes the piston 74 coupled to the rod 40. The optical source 28, in this embodiment includes a pair of laser diode bars 70a and 70b (collectively referred to as laser diode bars 70). The laser diode bars 70a and 70b include emitter surfaces 72a and 72b, respectively. The optical source 28 optionally includes optical elements 76a and 76b which are located between the emitter surfaces 72a and 72b and the radiation directing elements 20a and 20b, respectively and extend the length L 64 of the cavity 12. If a longer cavity 12 is desired, multiple diode bars can be combined. Heat sink 46 includes cooling fins 48 arranged in an array.

In operation, laser diode bars 72, provide continuous or pulsed optical radiation to skin drawn into cavity 12. It will be appreciated that other sources of optical radiation including but not limited to incandescent lamps, flashlamps, halogen lamps, light emitting diodes or any other suitable light source presently available or yet-to-be developed can be used to provide treatment radiation. These sources can be optionally combined with filters to provide one or more selected wavelengths or separate bands of wavelengths from about 380 nm to about 1350 nm. The optical radiation source or sources may also provide a fluence of between 0.1-100 J/cm², pulse widths of between 1-1000 ms, spot sizes of 0.5-10 cm², and rep rates of 0.2 Hz or continuous wave. It is to be understood that pulse widths can include individual pulses or groups of pulses applied to each section of skin treated within cavity 12 in stamping mode, or pulse width can be the effective pulse width seen by each section of skin treated within cavity 12 as the device is moved over the surface of the skin and different sections of skin are moved into and out of the cavity.

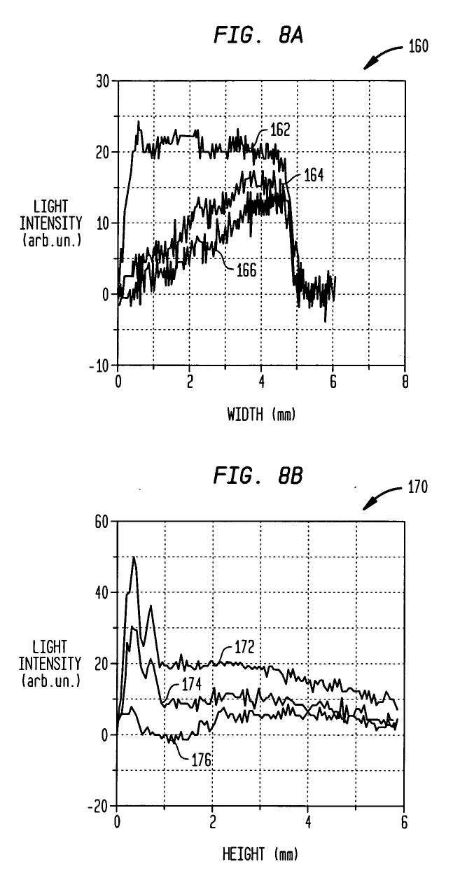

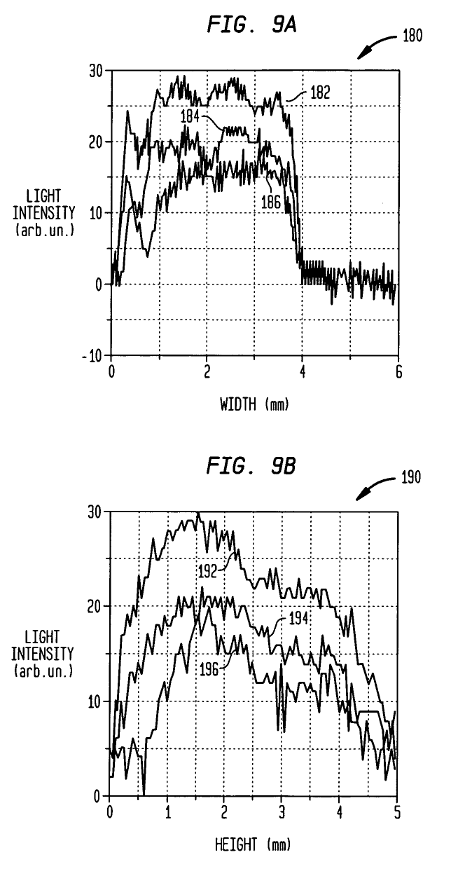

Optional optical elements 76a and 76b can focus, concentrate, diverge or collimate the radiation from optical radiation sources 72. The optical radiation sources 28 and optional optical elements 76a and 76b are aligned with radiation directing elements 20 such that optical radiation from each of the optical sources is coupled into radiation directing elements that then direct the light into cavity 12. As described above, the dimensions of cavity 12 are preferably chosen to allow the light from the different radiation directing elements to combine within the cavity in the area of the targets to be treated to provide improved efficacy while also providing an improved safety ratio to protect the epidermis. This is described more fully below in conjunction with FIGS. 8A, 8B, 9A and 9B.

Optionally, the radiation directing elements can deliver a pattern of treatment energy to the tissue within cavity 12 such that separate volumes of tissue within the cavity 12 are treated while surrounding tissue is untreated. That is, instead of uniformly treating all the tissue within cavity 12, only certain small volumes within the cavity may be treated. The healthy tissue in between these treated portions can improve healing time and tissue response to treatment. Such patterns of treatment may be provided by, for example, including focusing elements within the radiation directing elements or coating the internal walls of cavity 12 with a mask having openings such that light only passes through the opening. As another example, the internal walls of cavity 12 may be textured to provide such a pattern of treatment.

In one embodiment, the operator triggers the negative pressure within cavity 12 by pushing device 10 (FIG. 1) against skin. For example, housing 16 (FIG. 1) of device 10 can be slidable with respect to treatment head 14, such that when the operator places the treatment head 14 against skin and continues to push against curved section 18, housing 16 slides further towards the skin. When the operator stops pushing on curved section 18, the action of the housing 16 sliding away from the skin can work in conjunction with the cylinder 30, rod 40, piston 74 and reversing mechanism 44 to lower the pressure within the cavity 12 and, hence, gather skin into the cavity. As described above, sensors (not shown) may be included within cavity 12, such that when skin is detected within the cavity 12, the laser diode bars 70 are activated to provide treatment radiation to the skin within the cavity.

In order to provide cooling for the optical source and optical components, the cooling material 50, for example, chilled water may be circulated in the chamber 26 by means of the pump 54 and the conductor pipe 52. It will be appreciated that other cooling means may be used. For example, chamber 26 may house a phase transfer material (e.g., ice, wax) that changes phase as it absorbs heat from fins 48. Instead, device 10 may include a small fan to force air past fins 48. As another example, the heat sink may be thermally coupled to housing 16 such that heat is passed to the operator's hand during use and/or to the air.

In an alternative embodiment, parameters of device 10 may be changed to provide different treatment to skin drawn within cavity 12 or to provide different treatment to different types of skin (e.g., facial skin might be treated differently than back or underarm skin). For example, the dimensions of the cavity (e.g., cavity width, length and/or height), the pressure differential in the cavity, the position of an optical source, filters, fluence, pulse width and other parameters are adjustable. In yet another embodiment, the skin gathering implement 34 uses an adhesive force or pinching force applied in conjunction with piston 74 to gather skin into the cavity. In still another embodiment, ultrasound energy is used instead of optical radiation.

As described above, device 10 can include safety sensors. In one embodiment, such sensors are used to detect the presence of skin within cavity 12 and only then allow the emission of light within the cavity. In addition, safety sensors can be used to detect when skin is drawn too deeply within cavity 12 and prevent emission of light within cavity 12. This would prevent less firm skin and any anatomy located nearby from being exposed to the light from optical source 28. For example, the skin around a person's eye, including the eye lid, is generally very pliable. The light that is generated from optical source 28 may be such as to be not safe for use around the eye, as it might potentially injure the eye. For example, the light may be such that it can pass through the eye lid, be absorbed by melanin in the iris, and damage the eye. Consequently, one or more safety sensors can be located to detect skin within the cavity 12 at a height/depth which indicates that it may be skin around the eye thereby preventing device 10 from operating the light source.

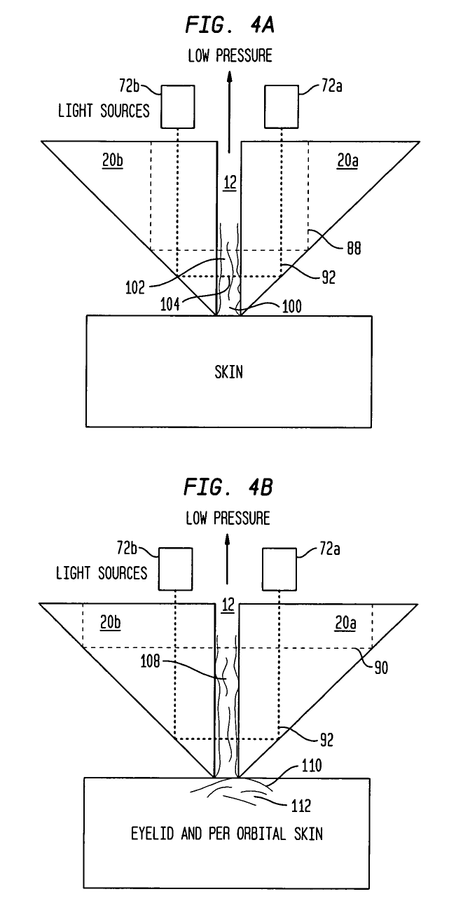

In one embodiment, safety sensors include one or more pairs of emitters and detectors. Referring to FIG. 3, treatment head 14 is shown to include emitter 84 and detector 86 which are aligned with radiation directing elements 20a and 20b, such that emitter 84 directs light along light path 88 which is then received by detector 86 when no skin is within cavity 12. When skin is drawn into cavity 12, light path 88 is interrupted and detector 86 sends a signal to control circuitry within electronic circuit boards 32a and 32b to allow the control circuitry to drive optical source 28 to emit light into cavity 12 to treat the skin. As shown, light path 88 is located close to the opening of cavity 12. However, light path 88 may be located deeper within (or at a greater height) within cavity 12 (though not as deep as light path 90) to indicate not only that the skin has been drawn within cavity 12 but that it has been drawn to a sufficient depth to permit treatment. This height is more fully described below with respect to FIGS. 4A and 4B.

In FIG. 3, treatment head 14 is also shown to include emitter 80 and detector 82 aligned to provide light path 90 which is deeper (or at a greater height) within cavity 12. Emitter 80 and detector 82 can be used to detect when skin is drawn too deeply within cavity 12, which as described above can indicate that this is skin that should not be treated. In this case, when light path 90 is interrupted, detector 82 sends a signal to the control circuitry to prevent the driving of optical source 28 such that the skin within the cavity is not treated.

In one embodiment photodiodes are used as detectors 82 and 86 and light emitting diodes (LEDs) are used as emitters 80 and 84. In another embodiment, treatment head 14 could include one or more reflectometer sensors (not shown) to detect the melanin content or other characteristics of the skin to be treated. Optionally treatment head 14 includes reflective surfaces 78 which allow optical radiation to enter the prisms 20a and 20b from optical source 28 but do not allow optical radiation scattered and reflected from the skin within cavity 12 back towards the optical radiation source 28 to escape from prisms 20a and 20b. Instead, reflective surfaces 78 return this scattered and reflected light back toward the skin in cavity 12 to improve the efficacy of treatment. This is referred to as “photon recycling”.

FIG. 4A shows a portion of skin to be treated 100 gathered into cavity 12 to a height sufficient to interrupt light path 88. In this example, the portion of the skin to be treated 100 includes one or more hair follicles and the treatment may be for hair removal. For simplicity, only hair shaft 102 and hair bulb 104 are shown. As described above, the height of skin within cavity 12 is a function of the width and shape of the cavity 12, the pressure differential applied to cavity 12, and the firmness of the skin. In this example, the hair follicles are treated by optical radiation from laser diode bars 70a and 70b delivered from opposite sides of cavity 12 by radiation directing elements 20a and 20b along path 92. As described above, the dimensions of cavity 12 and the optical radiation parameters are chosen such that the radiation from both radiation elements 20a and 20b is combined in hair follicles within the skin to improve the efficacy of treatment.

With regard to avoiding treating more pliable skin around the eye area, it has been discovered that an approximate relationship exists between the maximum height of the skin gathered into the cavity hₛₖᵢₙ₋ₘₐₓ and the width of the cavity w_{cavity} as follows:

hₛₖᵢₙ₋ₘₐₓ≈w_{cavity} for treatable skin (e.g., skin of sufficient firmness) and a nominal pressure differential of 20 cm of Hg (8 inches of Hg); and

Generally, hₛₖᵢₙ₋ₘₐₓ>2 w_{cavity} for non-treatable skin (e.g. less firm skin such as an eye lid) and a nominal pressure differential of 20 cm of Hg.

It has further been discovered that the range of skin height hₛₖᵢₙ is in an approximate range of:
0.5 w_{cavity}>hₛₖᵢₙ>2 w_{cavity}
The determination of hₛₖᵢₙ₋ₘₐₓ and hₛₖᵢₙ allows for the determination of the location of sensor light paths 88 and 90.

FIG. 4B shows that the skin 108 drawn within cavity 12 has been drawn in so deeply that it has interrupted light path 90, indicating that this is skin that is not to be treated, for example an eye lid. The structure of the iris 112 is such that the iris is not pulled into the cavity 12. As described above, less firm skin is gathered a much further distance (to a greater height) into the cavity 12. In one embodiment, the light path 90 is set to be interrupted when the skin height hₛₖᵢₙ is greater than 10 mm thereby causing detector 82 to send a signal to the control circuitry to prevent optical source 28 from being triggered.

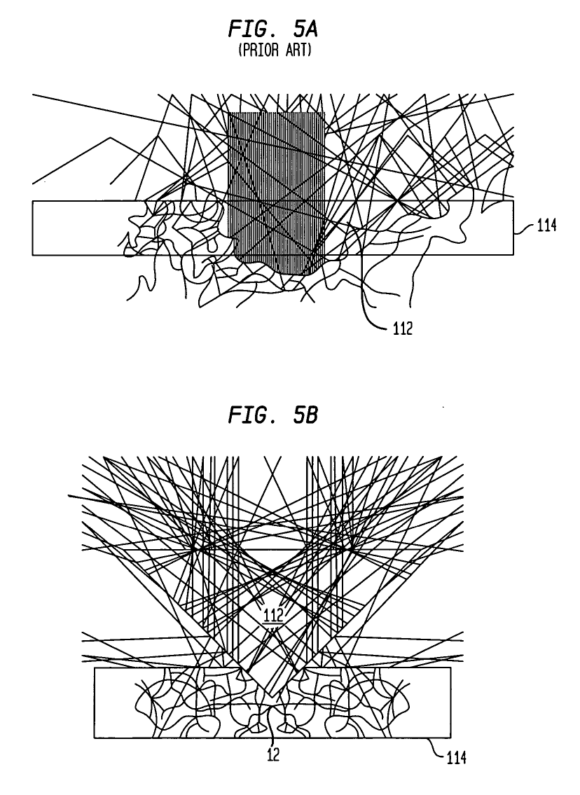

FIG. 5A is a diagram showing direct optical radiation being applied to a volume of skin 114′ by a prior art device. This diagram illustrates that significant radiation penetrates into volume 114′ and, if volume 114′ were an eye lid, such radiation would reach iris 112 through the eye lid. If this radiation is absorbed by melanin in the iris, it may damage the eye. In contrast, FIG. 5B is a diagram showing optical radiation being applied through radiation directing elements 20a and 20b to skin which has been drawn into cavity 12 and the significantly smaller amount of indirect light that may reach skin volume 114 outside of cavity 12. This diagram demonstrates, that even if device 10 did not have the safety sensors (e.g., 80, 82) described above for detecting the drawing into the cavity of skin around the eye, it would still provide significant eye safety as compared to prior art devices because it reduces the amount of light that could reach the iris.

FIG. 6 is a graph 130 of skin fold height vs. pressure for a cavity similar to the cavity 12 of FIG. 1. It has been determined that the dimensions of cavity 12 limit the volume of skin that can be gathered into cavity 12 such that increasing the pressure differential beyond the pressure necessary to draw that volume of skin into cavity 12 will not draw in more skin. Here point 134 on curve 132 represents an initial pressure of about 8 inches of Hg (20 cm of Hg) (200 Torr), which results in a skin height of about 5 mm in the cavity. Due to skin elasticity, the skin pulls back to a slightly lesser height, and as shown, increasing the pressure differential beyond point 134 does not increase the height of the gathered skin. Here, pressure differential refers to the pressure gradient between the volume in a cavity and the ambient (e.g. atmospheric pressure) outside the cavity.

It is advantageous to provide the minimum pressure differential to achieve a consistent skin height, preferably about 2 mm to about 6 mm, in the cavity for hair treatment and 1-3 mm for acne treatment. Using contoured edges at the entrance to the cavity and/or applying a lotion or oil to the skin surface to be treated.

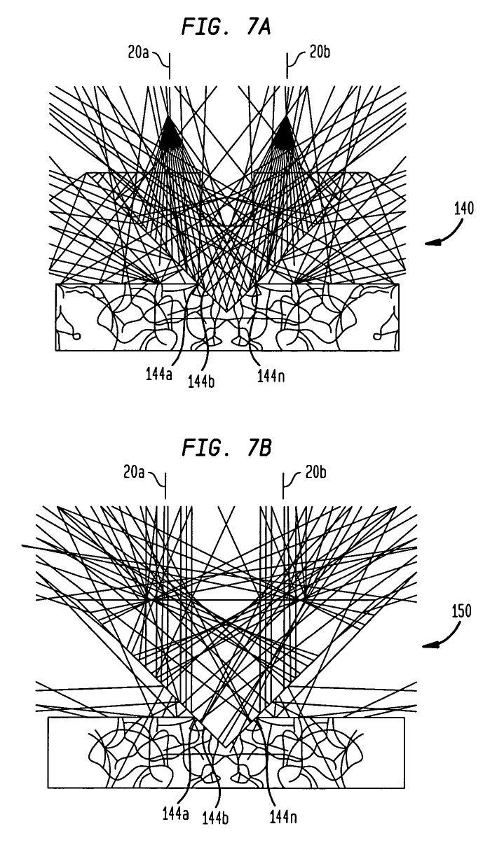

FIG. 7A represents treatment head 14 without reflector 78 (see also FIG. 3) and without reflective surfaces on the external surfaces of radiation directing elements 20a and 20b. In contrast, FIG. 7B represents treatment head 14 including reflector 78 and also reflective surfaces on the external surfaces of radiation directing elements 20a and 20b. That is, in FIG. 7B, radiation directing elements 20a and 20b have reflective surfaces except on the surfaces that form cavity 12. As shown, the device of FIG. 7B directs significantly more light to the skin drawn into cavity 12 than does the device of FIG. 7A. In one embodiment, the reflective surfaces, including reflector 78, are coatings applied to all the surfaces of radiation directing elements 20a and 20b except those surfaces that form or are coupled to cavity 12. Using reflective surfaces, the efficiency of the delivery of optical radiation to targets within cavity 12 may be increased 1.2-4 times as compared with not using such reflective surfaces. As shown in FIGS. 7A and 7B, the targets within the skin volume 140 drawn into cavity 12 may be follicles 144a-144n.

FIG. 8A, 8B, 9A and 9B are light distribution graphs for two experimental treatment heads similar to the treatment head 14 of device 10. In this device, the optical source or sources (e.g., diode lasers generating light of wavelength 800 nm) are coupled to cavity 12 through optical fibers. The light distribution graphs illustrate the importance of the cavity dimensions (e.g., width) and how proper selection of dimensions with alignment of the optical components allows light from different radiation directing elements to be combined or overlapped within cavity 12 for better efficacy and higher safety ratio.

FIG. 8A is a graph of light intensity versus cavity width, and each of curves 164 and 166 represent light emitted into a cavity from only one radiation directing element, in this case a fiber, at one side of the cavity (e.g., 5 mm). In this example, the width of the cavity is 5 mm and curves 164 and 166 show that the maximum light intensity is at the skin surface adjacent the cavity wall (e.g., at 5 mm). This results in a low safety ratio which can lead to epidermal injury. Curve 166 has reduced light intensity as compared to curve 164 because the reflector used for curve 166 was brown paper which absorbed more light than the more reflective surface used for curve 164. In contrast, curve 162 represents light being emitted from two radiation directing elements on opposite sides of the cavity and combining within the volume of the cavity such that the light intensity within the volume is substantially the same as the light at each of the surfaces of the cavity (0 mm and 5 mm). This results in a higher safety ratio such that targets may more easily be treated while protecting the epidermis. It is also possible to configure the cavity and/or radiation directing elements such that the amount of light received within the volume of skin within the cavity is higher than the amount of light received at the skin surface in contact with the cavity walls. In addition, the cavity walls can be cooled to cool the skin surface and provide additional epidermal safety.

FIG. 8B is a graph of light intensity versus cavity height, and again each of curves 174 and 176 represent light emitted into a cavity from only one radiation directing element and curve 172 represents light emitted from two radiation directing elements on opposite sides of a cavity. Curve 176 has reduced light intensity as compared to curve 174 again because the reflector used for curve 176 was brown paper which absorbed more light than the more reflective surface used for curve 174. In this example, the height to which skin is drawn within the cavity is 5 mm. As shown in curve 172, combining light from multiple radiation directing elements on different sides of the cavity provides increased light intensity at the same height as provided by light from only one radiation directing element (curves 174 and 176).

The graphs of FIGS. 9A and 9B are similar to the graphs of FIGS. 8A and 8B except that the cavity width and height are 4 mm.

It has been determined that the optimum cavity dimensions include a height which is larger than the depth of the target from the skin surface and a width that is less than four times the depth of the target from the skin surface, preferably less than 2 times the depth of the target from the skin surface.

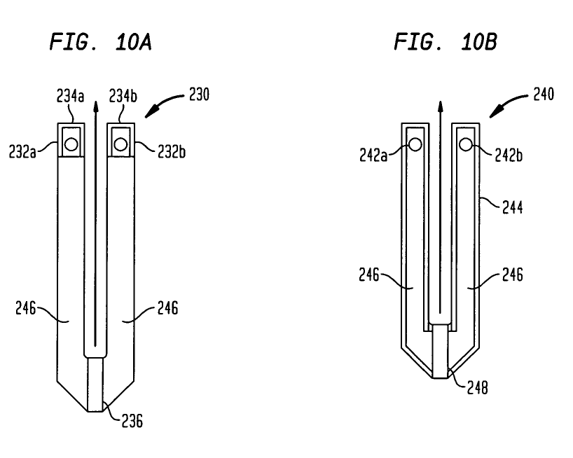

Referring to FIGS. 10A and 10B two alternative lamp based optical sources 230 and 240 are shown. Optical source 230 includes lamps 232a and 232b (collectively referred to as lamp 232) disposed adjacent reflectors 234a and 234b (collectively referred to as reflector 234), respectively. Each lamp 232 and reflector 234 combination operates similarly to the laser diode bars 70 of FIG. 3. In one embodiment, the lamp is a high efficiency Xe flashlamp. In this embodiment, lamp 232 operates with a fluence of about 0.1 to about 100 J/cm², a pulse width of about 1 ms to about 500 ms, a wavelength range of between 400-1350 nm and preferably between 600-1200 nm. The reflectors 232 include a reflective coating and external surfaces of prisms 246 may also have a reflective coating. Here the overall efficiency of the treatment head is approximately 10-40 percent. Optionally, a spectral filter can be incorporated in device 230. In one embodiment, such spectral filter can be a dielectric coating on the surfaces of prisms 246 that receive light from the lamps or a coating on the lamps themselves. The lamps can be cooled by air or liquid flow.

In the embodiment of FIG. 10B, lamps 242a and 242b are integrated within prisms 246. That is, cavities are made within the prisms such that the internal walls of these cavities provide the envelope for the lamps eliminating the need for a separate glass envelope for each lamp. In this embodiment, the overall efficiency of the treatment head is increased by approximately 150-250%. As described above a reflective coating can be provided on the external surfaces of the prisms 246.

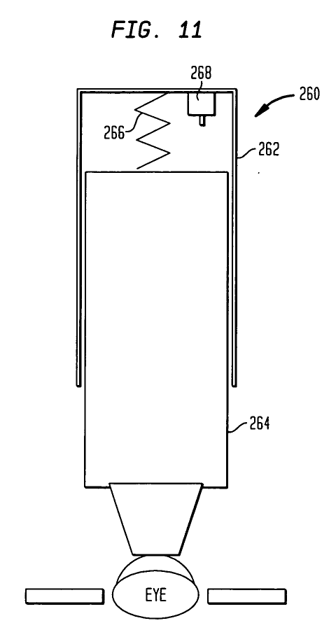

Referring to FIG. 11, a pressure controlled firing mechanism may be used to provide eye protection. Device 260 includes a housing 262 that is slideable over internal component 264, and housing 262 and internal component 264 are forced apart by a spring 266. Housing 262 includes a sensor 268, which may, for example, be a microswitch. In order for control circuitry to cause the optical radiation sources to emit light, a signal must be received from sensor 268. Sensor 268 is triggered when it is pressed against internal component 264. In operation, this occurs when device 260 is placed against skin (specifically the skin contacting surface of internal component 264 is placed against the skin) and sufficient force is applied to housing 262 to compress spring 266 and allow the housing to slide toward the skin. As the housing is moved toward the skin, it will bring sensor 268 into contact with internal component 264 and sensor 268 will send a signal to the control circuitry allowing it to trigger the radiation source(s). Because most treatable facial skin has bone behind, the treatable areas of facial skin can tolerate the pressure necessary to enable activation. However, because there is no bone behind the eye, it would be difficult and painful to place enough force on device 260 to enable activation, thereby providing eye protection. Spring 266 can be provided with an adjustable spring tension mechanism (not shown).

The pressure-controlled firing mechanism shown in device 260 can be combined with other safety features. In addition, the pressure controlled firing mechanism can be combined into device such as device 10 of FIG. 1 having a cavity within which skin may be drawn.

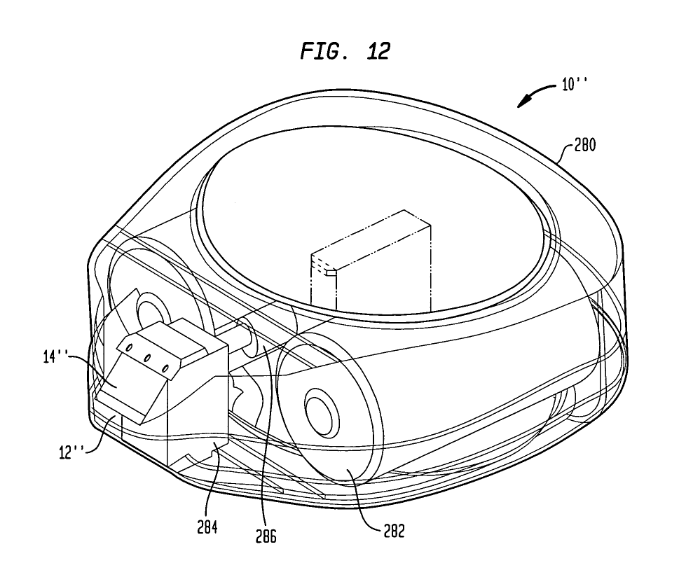

Referring to FIG. 12, a skin treatment device 10″ similar to the device 10 of FIG. 1 is shown. Device 10″ includes treatment head 14″, including a cavity 12″, power supply 282, cooling cartridge 284 thermally coupled to the treatment head 14″, and skin gathering implement 286 (partially shown).

Device 10″ operates in a manner similar to device 10. In one embodiment, the power supply 282 is one or more high capacity rechargeable batteries or capacitors. In a home use application, the power supply 282 could provide power for about two to about five minutes of operation, or longer depending on the desired duration of the treatment. The removable cooling cartridge 284 provides cooling for the optical sources and other optical components, for example, radiation directing elements in treatment head 14″. The removable cooling cartridge 284 may act as chamber 26 including material 50 to provide cooling a heat sink, such as heat sink 46 of FIG. 1. In one embodiment, the removable cooling cartridge 284 can be placed in a household freezer before being used.

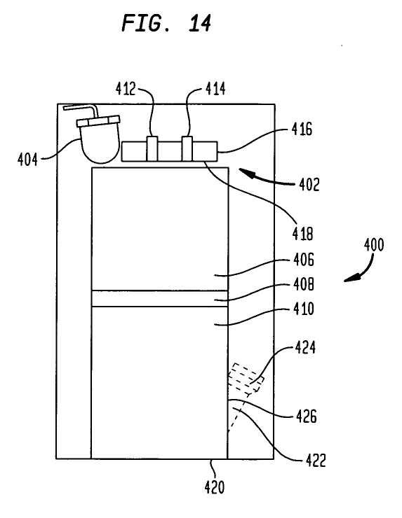

Referring to FIG. 14, in another embodiment, a dermatological treatment device utilizes a treatment radiation delivery system 400 that is configured to treat tissue, such as the skin, while ensuring that the radiation transmitted to the tissue is both eye and skin safe. Thus, system 400 will not damage the eye, tissues in the eye, or other tissues. System 400 can be designed or integrated as part of photocosmetic devices for home or professional or other uses.

System 400 includes a treatment radiation source 402, an eye-safety radiation source 404, a waveguide 406, a diffuser 408 and a contact element 410. Treatment radiation source 402 is a laser diode bar having two laser diodes 412 and 414. However, many other possible configurations of treatment radiation source 402 are possible, such as solid state lasers, incoherent sources (i.e. lamps of various types), etc. Additionally, different configurations of laser diode bars in particular are possible and potentially preferable depending on the application and the design specifications. For example, the number of radiation sources can be varied and positioned to provide the required radiation power at the skin and to provide a homogenized distribution of treatment radiation throughout the waveguide and at the transition from the device to the tissue being treated. The optimal design configuration(s) will depend on a number of variables, including the type of treatment, the size of the device, the spot size of the treatment, the materials being used, the wavelength(s) of radiation selected for the treatment, etc.

In treatment radiation delivery system 400, laser diodes 412 and 414 are mounted in a substrate 416. Substrate 416 is made of copper, but could alternatively be made of silicon carbide, copper tungsten, or other suitable materials. Substrate 416 provides mechanical stability and removes waste heat during operation. Preferably, the surface 418 of substrate 416 is coated with a material that is highly reflective of the treatment-radiation wavelength to recycle photons scattered/reflected from the skin. Recycling photons both reduces the heat load on substrate 416 (and system 400 generally), and it improves treatment efficacy.

Waveguide 406 channels the treatment radiation and homogenizes the spatial profile of the treatment radiation to more evenly distribute the treatment radiation that is transmitted to the tissue. Treatment radiation from treatment radiation source 402 is coupled into waveguide 406. The input surface of waveguide 406 can be, for example, anti-reflection coated or bonded to the radiation source 402 to prevent radiation loss.

Alternatively, radiation source 402 may be protected by a window between surface 418 and waveguide 406. Such a window may be configured, for example, to reduce the exposure of radiation source 204 to reflected radiation. The inner surface of the window (i.e. the portion facing radiation source 402) can be anti-reflection coated, while the outer surface (i.e., the portion facing waveguide 406) can either be anti-reflection coated or bonded to the input surface of waveguide 406.

Diffuser 408 is located on the side of the waveguide opposite treatment radiation source 402. Diffuser 408 is typically made of glass, plastic, or other optical material. Diffuser 408 increases the angular spectrum of the treatment radiation at each point within the radiation beam. For a fixed treatment having radiation output power that is limited to a predetermined maximum level, the diffuser can be designed to prevent retinal damage to the subject being treated by increasing the angular spectrum to the point where the output beam meets the ANSI eye safety standards. Many different radiation diffusers can be used including, but not limited to, holographic, diffractive, photolithographic, fiber bundle, milk glass, sandblasted glass, or other suitable material. Some diffusers that are suitable for use in embodiments similar to system 400 may have a textured output surface. For some of those diffusers, a space (e.g., filled with air or a fluid) may be required between the diffuser and the contact element, if the device includes a contact element 410. Volume diffusers may be used with or without an air gap.

In system 400, the radiation exiting diffuser 408 is coupled directly into contact element 410. Contact element 410 serves several functions. Contact element 410 acts as a waveguide to couple the radiation to the skin. The treatment radiation exits device 400 through contact surface 420. The length of contact element 410 preferably is chosen so as to create a uniform radiation distribution at the skin surface. In system 400, the length of contact element 410 can be adjusted based on the design parameters to optimize the device, but typically would be in the range of 0.5-100 mm. Contact element 410 also provides contact cooling during treatment. Contact element 410 is made of a thermally conductive transparent material, in this case sapphire. However, other substances can be used. Heat can be removed from contact element 410 by, for example, attaching (with glue or other suitable means) a metal heat exchanger to the exterior surface of contact element 410. The metal heat exchanger can be coated with a highly reflective coating, so that any treatment radiation that is not totally internally reflected at the sapphire/glue interface is not absorbed.

Eye safety radiation source 404 is an LED located at the top surface of waveguide 404. Source 404 provides radiation at a wavelength that is chosen to maximize the perceived brightness by the user after the radiation propagates through the eyelid and the anterior portion of the eye. In other words, the wavelength is preferably a wavelength that is irritating to the subject being treated but is generally safe even at intensities perceived by the subject being treated to be painful or harmful. Source 400 emits light at wavelengths in the red range (600-680 nm), and at a power density of 1-10 mW/cm². Other wavelengths and intensities are possible, however, depending on the design and specifications.

After the source 404 has been engaged and contact surface 420 has been in contact with the tissue being treated for approximately 1.0-2.0 seconds, treatment source 402 is engaged and the tissue is irradiated. The 1.0-2.0 second time period is chosen so the user has sufficient time to remove the device from her eye prior to irradiation by the treatment source 402. However, other embodiments are possible. For example, a shorter time or longer time could be used, such as 0.1 to 3.0 seconds. A shorter time period could be used, for example, to allow time for a quicker aversive response to occur, such as the twitch or squint of an eye, that would indicate to a person treating the subject that the eye has been irradiated. The aversive response may be any movement that causes the subject to move the device from the tissue, or any movement that indicates to a person treating the subject that the eye may be irradiated, including, without limitation, squinting, pupil dilation, eye movement, head movement, and arm movement.

The existence of contact with the tissue being treated can be determined by a number of different contact sensors. System 400 also utilizes source 404 as part of a contact sensing mechanism in addition to the “pre-pulse” safety mechanism described in the paragraph above. The contact sensing mechanism includes source 404, prism 422 and detector 424. Prism 422 is mounted at an angle along on outer side surface of contact element 410. Prism 422 is optically coupled to contact element 410 to allow light to pass from contact element 410 and through prism 422. Detector 424 is attached to an end surface of prism 422 and is optically coupled to prism 422 to receive light passing from contact element 410 and through prism 422.

System 400 determines whether surface 420 is in contact with tissue by transmitting radiation from source 404 through treatment radiation delivery system 400 to contact surface 420. When contact surface 420 is in contact with the skin, there is only a very small background signal at detector 424 due to total internal reflection at coupling prism interface 426. When contact surface 420 is in contact with tissue, the amount of radiation coupled out of contact element 410 via prism 422 and into detector 424 increases significantly due to the scattering of light from the skin to the coupling prism interface 426 at angles that are not internally reflected within contact element 410. The output of detector 424 is monitored by control electronics of the device (not shown), and, when the voltage exceeds pre-determined thresholds, the device determines that contact surface 420 is in contact with the tissue being treated. Thus, detector 424 can serve as an aversive sensor by detecting aversive motion of the patient relative to the device.

To facilitate the dual purposes of source 404, source 404 is a bicolor LED with one wavelength for contact sensing (in system 400, in the near infrared range) and one wavelength for the “pre-pulse” safety mechanism (in system 400, in the red range as discussed above). Preferably, the wavelengths used for the contact sensing mechanism and the “pre-pulse” safety mechanism will be different than the primary wavelength(s) used for treatment, although this is not essential. The first wavelength of source 404 is applied to sense contact. After contact with the tissue has been detected for a certain minimum time (typically 50 ms), the second wavelength is applied to warn the subject that the laser is about to fire. If the device is aimed at the eye, the light from the second wavelength will severely irritate (but not damage) the eye. Even if the system is in contact with a closed eyelid, the second wavelength is at such an intensity that the subject will still react to the light by turning her head or pulling the device away. At that point, the contact sensing mechanism determines that contact surface 420 is no longer in contact with the tissue and the device will not irradiate the tissue.

Although system 400 is designed for use while in contact with the tissue to be treated other embodiments are possible. For example, an alternate embodiment could utilize proximity sensors to operate near, but not in contact with, the tissue. The device could also eliminate all such sensors and could be designed to operate at some distance from the tissue (or to operate while in contact with the tissue without utilizing a contact sensor). Additionally, the cooling provided by contact element 410 could be provided by other mechanisms (such as a cryogenic spray, a separate cooling plate, pre-cooling the tissue, or by other mechanisms). Furthermore, although system 400 is primarily designed for use with optical wavelengths of light, many other wavelengths or combinations of wavelengths (both optical and otherwise) are possible.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

What is claimed is:

1. A method for treating a tissue of a subject with radiation in an eye-safe manner, comprising:
irradiating said tissue with an eye-safe radiation having a wavelength and intensity chosen to cause an aversive response by said subject when said eye-safe radiation irradiates said subject's eye;
waiting a predetermined period of time; and
irradiating said tissue with a treatment radiation when said aversive response does not occur within said period of time;
wherein said tissue is not irradiated with said treatment radiation when said aversive response does occur within said period of time.

2. The method of claim 1, wherein said eye-safe radiation has a wavelength in the range of 600-680 nm.

3. The method of claim 1, wherein said eye-safe radiation has a wavelength that is predominately red.

4. The method of claim 1, wherein said eye-safe radiation has an intensity in the range of 1-10 mW/cm².

5. The method of claim 1, wherein said period of time is in the range of approximately 0.1 to 3.0 seconds.

6. The method of claim 1, wherein said period of time is in the range of approximately 1.0 to 2.0 seconds.

7. The method of claim 1, further comprising determining whether said aversive response has occurred.

8. The method of claim 7, further comprising inhibiting the transmission of said treatment radiation when said aversive response has occurred.

9. The method of claim 1, further comprising contacting said tissue with an applicator to transmit said eye-safe radiation.

10. The method of claim 9, wherein said tissue is irradiated with said eye-safe radiation only if said applicator is in contact with said tissue.

11. The method of claim 9, wherein said tissue is irradiated with said treatment radiation only if said applicator is in contact with said tissue.

12. The method of claim 1, further comprising orienting an applicator to irradiate said tissue with said eye-safe radiation.

13. The method of claim 12, wherein said tissue is irradiated with said eye-safe radiation only if said applicator is in proximity of said tissue.

14. The method of claim 12, wherein said tissue is irradiated with said treatment radiation only if said applicator is in proximity of said tissue.

15. An apparatus for treating tissue with radiation in an eye-safe manner, comprising:
a controller for controlling the production of radiation and configured to provide first and second control signals;
a first radiation source configured to produce in response to said first control signal an eye-safe radiation at an intensity that irritates a subject's eye;
a second radiation source configured to produce in response to said second control signal a treatment radiation;
a radiation transmission path configured to transmit radiation from said first radiation source to said tissue through a radiation transmission surface;
a sensor in electrical communication with said controller and configured to provide a sensor signal when said radiation transmission surface is in proximity to said tissue;
wherein said controller is configured to provide said second control signal after a predetermined time interval following said first control signal and when said sensor signal indicates that said radiation transmission surface remains in proximity to said tissue.

16. The apparatus of claim 15, wherein said first radiation source is a diode.

17. The apparatus of claim 15, wherein said first radiation source is configured to produce radiation in the range of 600-680 nm.

18. The apparatus of claim 15, wherein said first radiation source is configured to produce radiation having a wavelength that is predominately red.

19. The apparatus of claim 15, wherein said first radiation source is configured to produce radiation having an intensity in the range of 1-10 mW/cm².

20. The apparatus of claim 15, wherein said predetermined time interval is in the range of approximately 0.1 to 3.0 seconds.

21. The apparatus of claim 15, wherein said predetermined time interval is in the range of approximately 1.0 to 2.0 seconds.

22. The apparatus of claim 15, wherein said controller is configured to provide said second control signal when said radiation transmission surface is in contact with said tissue.

23. The apparatus of claim 15, wherein said controller is configured to provide said first control signal when said radiation transmission surface is in contact with said tissue.

24. The apparatus of claim 15, wherein said sensor is configured to detect an aversive response from said subject in response to said eye-safe radiation.

25. The apparatus of claim 15, wherein said aversive response is one of squinting, pupil dilation, eye movement, head movement, and arm movement.

26. The apparatus of claim 15, wherein said first radiation source is further configured to provide sensor radiation, and wherein said sensor is a detector configured to detect said sensor radiation.

27. The apparatus of claim 26 wherein said sensor radiation has a wavelength in the near infrared range.

28. The apparatus of claim 26 wherein said detector is configured to provide said sensor signal when said sensor radiation exceeds a first predetermined threshold.

29. The apparatus of claim 26, wherein said radiation transmission path is configured to substantially totally internally reflect said sensor radiation when said radiation transmission surface is not in contact with said tissue.

30. The apparatus of claim 15, wherein said radiation transmission path is configured to substantially totally internally reflect said eye-safe radiation when said radiation transmission surface is not in contact with said tissue.

31. The apparatus of claim 15, wherein said radiation transmission path is configured to substantially totally internally reflect said treatment radiation when said radiation transmission surface is not in contact with said tissue.

32. The apparatus of claim 15, wherein said radiation transmission path further comprises:
a first waveguide section;
a second waveguide section; and
a diffuser;
wherein said first waveguide section is located between said first source and said diffuser and said second waveguide section is located between said diffuser and said radiation transmission surface.

33. The apparatus of claim 32, wherein said diffuser extends across substantially the entire said radiation transmission path.

34. The apparatus of claim 32, wherein said diffuser is made of at least one of plastic, glass, and sapphire.

35. The apparatus of claim 32, wherein said second waveguide section is sapphire.

36. The apparatus of claim 32, wherein said second waveguide section includes a cooling mechanism configured to cool said tissue.

37. The apparatus of claim 15, wherein said radiation transmission path includes a cooling mechanism configured to cool said tissue.

38. The apparatus of claim 15, wherein said radiation transmission path is made substantially of sapphire.

39. The apparatus of claim 15, wherein said radiation transmission path includes a diffuser extending across a portion of said radiation transmission path and oriented to diffuse radiation produced by said second radiation source.

40. An apparatus for treating tissue with radiation in an eye-safe manner, comprising:
a radiation source assembly in electrical communication with a controller;
a waveguide configured to transmit radiation from said radiation source assembly to said tissue;
a sensor in electrical communication with said controller and configured to provide a sensor signal when said radiation transmission surface is in proximity to said tissue;
wherein said radiation source assembly is configured to provide in response to signals from said controller a first radiation that is eye-safe and of an intensity capable of causing an aversive reaction from a subject when irradiating the subject's eye, a second radiation that is capable of treating said tissue, said second radiation being provided a predetermined time after said first radiation when said sensor indicates that said waveguide remains in proximity of said tissue.

41. The apparatus of claim 40, wherein said radiation source assembly is further configured to provide a third radiation, wherein said sensor is configured to detect said third radiation and issue a sensor signal based on the level of radiation detected.

42. The apparatus of claim 40, wherein said waveguide further includes a diffuser extending across a portion of said waveguide and oriented to diffuse radiation produced by said radiation source assembly.

43. The apparatus of claim 40, wherein said waveguide includes a cooling mechanism configured to cool said tissue.

44. The apparatus of claim 40, wherein said sensor is configured to provide a sensor signal only when said radiation transmission surface is in contact with said tissue.

45. An apparatus for photocosmetic treatment of a subject's tissue comprising:
a pressure source;
a cavity having an open end, said cavity in fluid communication with said pressure source, and said open end configured to receive said tissue when said pressure source applies pressure;
at least one radiation source configured to transmit radiation into said cavity; and
a sensor configured to issue a sensor signal;
wherein said sensor signal prevents the transmission of radiation from said radiation transmission source when said sensor detects tissue that is not suitable for treatment.

46. The apparatus of claim 45, wherein said radiation source is configured to transmit radiation from at least two different directions within said cavity.

47. The apparatus of claim 45, wherein said radiation source is configured to treat a set of two or more volumes of tissue each separated by untreated tissue.

48. The apparatus of claim 45, wherein said radiation source is configured to provide radiation to an array of independent treatment sites within said cavity, wherein each such treatment site is separated by untreated tissue within said cavity.

49. The apparatus of claim 45, wherein said sensor is a pressure sensor.

50. The apparatus of claim 45, wherein said sensor is a depth sensor configured to sense a depth of said tissue within said cavity.

51. The apparatus of claim 50, wherein said sensor is configured to provide a control signal inhibiting the transmission of radiation by said radiation source when said tissue extends beyond a predetermined depth into said cavity.

52. The apparatus of claim 45, wherein said sensor is a radiation intensity sensor.

53. The apparatus of claim 52, wherein said radiation intensity sensor is configured to provide a control signal inhibiting the transmission of radiation by said radiation source when said radiation exceeds a predetermined threshold.

54. The apparatus of claim 52, wherein said radiation intensity sensor is configured to provide a control signal inhibiting the transmission of radiation by said radiation source when said radiation is substantially totally internally reflected.

55. The apparatus of claim 45, wherein said apparatus is configured to operate within a predetermined safety ratio.

56. The apparatus of claim 45, wherein said cavity has a depth that is greater than the depth of a target in said tissue to be treated from the surface of said tissue to be treated.

57. The apparatus of claim 45, wherein said cavity has a side that is less than four times the depth of a target in said tissue to be treated from the surface of said tissue to be treated.

58. The apparatus of claim 45, wherein said radiation source is configured to irradiate said tissue at a fluence of about 0.1 to about 100 J/cm².

59. The apparatus of claim 45, wherein said radiation source is configured to irradiate said tissue at a pulse width of about 1 ms to about 500 ms.

60. The apparatus of claim 45, wherein said radiation source is configured to irradiate said tissue at a wavelength range of between approximately 400-1350 nm.

61. The apparatus of claim 45, wherein said radiation source is configured to irradiate said tissue at a wavelength range of between approximately 600-1200 nm.

62. A method for photocosmetic treatment of a subject's tissue comprising:
drawing a volume of said tissue into a cavity;
determining whether said volume of tissue is safe to treat using radiation; and
treating said volume of tissue with radiation based on said determination;
wherein said volume of tissue is not treated if it is determined that said tissue is unsafe to treat, and wherein said volume of tissue is treated if it is determined that said tissue is safe to treat.

63. The method of claim 62, wherein said treating includes transmitting radiation from at least two different directions.

64. The method of claim 63, wherein said radiation from at least two different directions overlaps at one or more targets on the skin.

65. The method of claim 63, wherein said radiation from at least two different directions treats a set of two or more volumes of tissue each surrounded by untreated tissue.

66. The method of claim 62, further comprising providing an array of independent treatment sites within said volume of tissue, wherein each such treatment site is separated by untreated tissue within said volume.

67. The method of claim 62, wherein said step of determining further comprises sensing a pressure applied to said tissue.

68. The method of claim 67, wherein said tissue is safe to treat if said pressure exceeds a predetermined threshold.

69. The method of claim 62, wherein said step of determining further comprises sensing the depth of said volume of said tissue within said cavity.

70. The method of claim 69, wherein said tissue is not safe to treat if said volume exceeds a predetermined depth within said cavity.

71. The method of claim 62, wherein said step of determining further comprises sensing radiation using a radiation intensity sensor.

72. The method of claim 71, wherein said tissue is not safe to treat when said radiation exceeds a predetermined threshold.

73. The method of claim 71, wherein said tissue is not safe to treat when said radiation is substantially totally internally reflected.

74. The method of claim 62, wherein said step of determining further comprises determining a ratio of a rise in temperature of the skin versus a rise in temperature of the target, and inhibiting the transmission of radiation with said ratio is not within predetermined limits.

75. The method of claim 62, wherein said step of treating further comprises irradiating said tissue at a fluence of about 0.1 to about 100 J/cm².

76. The method of claim 62, wherein said step of treating further comprises irradiating said tissue with a pulse width of about 1 ms to about 500 ms.

77. The method of claim 62, wherein said step of treating further comprises irradiating said tissue with at least one wavelength in the range of between approximately 400-1350 nm.

78. The method of claim 62, wherein said step of treating further comprises irradiating said tissue with at least one wavelength in the range of between approximately 600-1200 nm.
